(19) United States
(12) Patent Application Publication
(10) Pub. No.: US 2020/0397465 A1
(43) Pub. Date: Dec. 24, 2020

(54) CONTROL APPARATUS FOR MEDICAL DEVICE AND METHOD OF CONTROLLING FLOW RATE OF FLUID

(71) Applicant: TERUMO KABUSHIKI KAISHA, Tokyo (JP)

(72) Inventors: Taiga NAKANO, Cupertino, CA (US); Junichi KOBAYASHI, Cupertino, CA (US); Tomonori HATTA, San Jose, CA (US); Kosuke NISHIO, Tokyo (JP)

(73) Assignee: TERUMO KABUSHIKI KAISHA, Tokyo (JP)

(21) Appl. No.: 17/009,910

(22) Filed: Sep. 2, 2020

Related U.S. Application Data
(63) Continuation of application No. PCT/JP2019/011689, filed on Mar. 20, 2019.

(30) Foreign Application Priority Data
Mar. 29, 2018 (JP) 2018-064009

(51) Int. Cl. A61B 17/3207; A61B 90/00

(52) U.S. Cl. CPC A61B 17/320758; A61B 90/03; A61B 2562/0247; A61B 2090/033; A61B 2217/007

(57) ABSTRACT

A control apparatus and a control method which are capable of suppressing appearance of a thrombus inside a medical device during a period other than the treatment period. The medical device includes a treatment unit configured to treat an object in a biological lumen. The control apparatus includes an aspiration source or a liquid delivering source connected to the medical device, and a control unit configured to control a flow rate of a fluid flowing inside the medical device. The control unit causes the fluid to flow inside the medical device at a constant flow rate during a treatment period of the treatment unit, and causes the fluid to flow inside the medical device at a flow rate lower than the constant flow rate during a period other than the treatment period, while the medical device is inserted into a living body.

### CROSS-REFERENCES TO RELATED APPLICATIONS

This application is a continuation of International Application No. PCT/JP2019/011689 filed on Mar. 20, 2019, which claims priority to Japanese Application No. 2018-064009 filed on Mar. 29, 2018, the entire content of both of which is incorporated herein by reference.

### FIELD OF THE DISCLOSURE

The present disclosure generally relates to a control apparatus for a medical device that causes a fluid to flow in an elongated tubular medical device configured to be inserted into a living body, and a method of controlling a flow rate of a fluid.

### BACKGROUND DISCUSSION

Medical devices are known having an elongated tubular shaft portion configured to be inserted into a living body and a treatment unit disposed in a distal portion of the shaft portion. For example, the medical device may include an atherectomy device in which a rotary body rotated by a drive shaft disposed in a shaft portion is used as a treatment unit so that a stenosed substance such as a thrombus is cut by rotating the rotary body inside a biological lumen. In the atherectomy device, in order to remove the cut stenosed substance, an aspiration source such as a pump or a syringe for aspirating a fluid inside the biological lumen is connected to the shaft portion. In addition, a liquid delivering source such as a pump or a syringe for delivering the fluid into the biological lumen is connected to the shaft portion.

In the atherectomy device, while the drive shaft is rotated, blood inside the shaft portion is aspirated by the aspiration source, and a saline solution (i.e., saline) is delivered into the shaft portion from the liquid delivering source. U.S. Patent Application Publication No. 2004/023162, for example, discloses a medical device.

In a state where a medical device is inserted into a living body, blood is present inside the medical device. The blood can stagnate inside the medical device when a treatment unit is not performing a treatment, such as when a treatment unit is being delivered to a target site. For this reason, there is a possibility that a thrombus may appear inside the medical device. When the thrombus appears inside the medical device, there are following risks. An aspiration force generated by an aspiration source can be weakened, and the appearing thrombus can cause a peripheral embolism. Rotation of a drive shaft can be disturbed, thereby degrading a treatment effect. In addition, the weakened aspiration force in the medical device may reduce efficiency in extracorporeally discharging a biological tissue piece produced as a result of the treatment performed by the treatment unit. Consequently, there is an increasing risk of the peripheral embolism caused by the biological tissue piece.

### SUMMARY

A control apparatus is disclosed for a medical device and a method of controlling a flow rate of a fluid, which can suppress appearance of a thrombus inside the medical device during a period other than the treatment period.

A control apparatus is disclosed for an elongated tubular medical device configured to be inserted into a living body having a distal portion in which a treatment unit for treating an object in a biological lumen is disposed. The control apparatus for the medical device includes an aspiration source or a liquid delivering source connected to the medical device, and a control unit that controls a flow rate of a fluid flowing inside the medical device. In a case where the aspiration source is connected to the medical device, the control unit controls the aspiration source so that the fluid inside the medical device is aspirated at a first flow rate during a treatment period of the treatment unit, and so that the fluid inside the medical device is aspirated at a second flow rate lower than the first flow rate during a period other than the treatment period, while the medical device is inserted into the living body, or in a case where the liquid delivering source is connected to the medical device, the control unit controls the liquid delivering source so that the fluid is delivered into the medical device at a third flow rate during the treatment period of the treatment unit, and so that the fluid is delivered into the medical device at a fourth flow rate lower than the third flow rate during the period other than the treatment period, while the medical device is inserted into the living body.

In addition, according to another aspect of the present disclosure, a control apparatus is disclosed for an elongated tubular medical device configured to be inserted into a living body and having a distal portion in which a treatment unit for treating an object in a biological lumen is disposed. The control apparatus for the medical device includes an aspiration source connected to the medical device, a liquid delivering source connected to the medical device, and a control unit that controls a flow rate of a fluid flowing inside the medical device. The control unit controls the aspiration source so that the fluid inside the medical device is aspirated at a first flow rate during a treatment period of the treatment unit, and so that the fluid inside the medical device is aspirated at a second flow rate lower than the first flow rate during a period other than the treatment period, while the medical device is inserted into the living body, and the control unit controls the liquid delivering source so that the fluid is delivered into the medical device at a third flow rate during the treatment period of the treatment unit, and so that the fluid is delivered into the medical device at a fourth flow rate lower than the third flow rate during the period other than the treatment period, while the medical device is inserted into the living body.

According to another aspect of the present disclosure, a method of controlling a flow rate of a fluid is disclosed. The method includes connecting a liquid delivering source to a medical device, the liquid delivering source configured to deliver the fluid into the medical device having a treatment unit disposed in a distal portion, or connecting an aspiration source configured to aspirate the fluid to the medical device, actuating a control unit configured to control the flow rate of the fluid to cause the fluid to flow in the liquid delivering source or the aspiration source at a constant flow rate while the medical device is stopped, and actuating the control unit to cause the fluid to flow in the liquid delivering source or the aspiration source at a flow rate higher than the constant flow rate while the medical device is actuated.

According to the control apparatus for the medical device which is configured as described above, the fluid flows inside the medical device even during the period other than the treatment period, while the medical device is inserted into the living body. Therefore, it is possible to suppress appearance of a thrombus inside the medical device.

According to the method of controlling the flow rate of the fluid which is configured as described above, the liquid delivering source or the aspiration source is controlled to cause the fluid to flow at the constant flow rate even while the medical device is stopped. Therefore, it is possible to suppress appearance of the thrombus inside the medical device.

### BRIEF DESCRIPTION OF THE DRAWINGS

FIG. 1 is a front view illustrating a whole medical device system according to the present embodiment.

FIG. 2 is a configuration diagram of the medical device system.

FIG. 3 is a cross-sectional view of a medical device.

FIG. 4 is a flowchart of treatment performed by the medical device system.

FIG. 5 is a time chart of each rotation speed of an aspiration pump and a liquid delivering pump in the medical device system.

FIGS. 6A and 6B are views illustrating a time chart and a modification example of each rotation speed at a low flow rate of the aspiration pump and the liquid delivering pump.

FIGS. 7A and 7B are views illustrating another modification example of the time chart of each rotation speed at the low flow rate of the aspiration pump and the liquid delivering pump.

FIG. 8 is a view illustrating another modification example of the time chart of each rotation speed at the low flow rate of the aspiration pump and the liquid delivering pump.

FIG. 9 is a cross-sectional view illustrating a periphery of an operation unit having a sensor unit including a pressure sensor.

FIG. 10 is a front view illustrating the periphery of the operation unit having the sensor unit including a switch.

### DETAILED DESCRIPTION

Set forth below with reference to the accompanying drawings is a detailed description of embodiments of a control apparatus for a medical device that causes a fluid to flow in an elongated tubular medical device configured to be inserted into a living body, and a method of controlling a flow rate of a fluid representing examples of the inventive control apparatus and method of controlling the flow rate. Note that since embodiments described below are preferred specific examples of the present disclosure, although various technically preferable limitations are given, the scope of the present disclosure is not limited to the embodiments unless otherwise specified in the following descriptions. Moreover, in the drawings, the same reference signs are assigned to the same components, and detailed descriptions of the same reference signs assigned to the same components are appropriately omitted. Dimensional ratios in the drawings may be exaggerated and different from actual ratios for convenience of description. In this specification, a side on which a medical device 10 is inserted into a biological lumen will be referred to as a “distal end” or a “distal side”, and an operator's hand-side will be referred to as a “proximal end” or a “proximal side”. A medical device system according to the embodiment of the present disclosure is used in treatment for cutting a stenosed site or an obstructive site caused by a plaque or a thrombus inside a blood vessel.

The medical device system includes the medical device 10 configured to be inserted into a living body and a control apparatus 11 configured to cause a fluid to flow inside the medical device 10. The medical device 10 may be, for example, an atherectomy device that cuts the stenosed site or the obstructive site inside the blood vessel by performing a rotary operation.

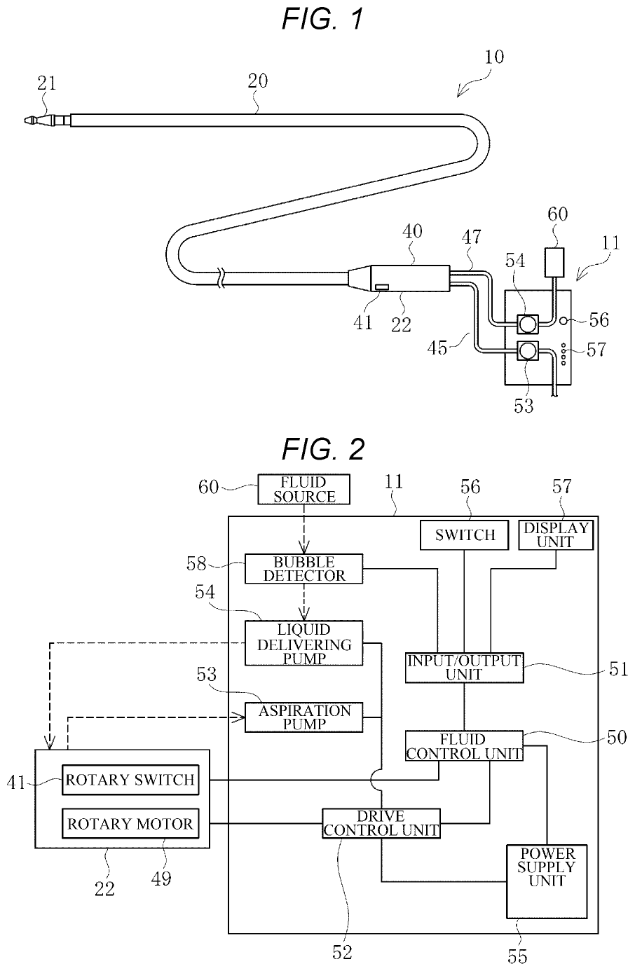

As illustrated in FIG. 1, the medical device 10 has an elongated long shaft portion 20, a rotatable treatment unit 21 disposed in a distal portion of the shaft portion 20, and an operation unit 22 disposed in a proximal portion of the shaft portion 20. The operation unit 22 has a housing 40 held and operated with a hand by an operator, and the housing 40 has a rotary switch 41 that performs operations for starting and stopping the rotation of the treatment unit 21.

The control apparatus 11 has an aspiration pump 53 serving as an aspiration source that aspirates fluid from the medical device 10, and a liquid delivering pump 54 serving as a liquid delivering source that delivers the fluid to the medical device 10. The aspiration pump 53 is connected to the medical device 10 by an aspiration tube 45 so that the fluid can flow between the aspiration pump 53 and the medical device 10. In addition, the liquid delivering pump 54 is connected to the medical device 10 by a liquid delivering tube 47 so that the fluid can flow between the liquid delivering pump 54 and the medical device 10. The aspiration pump 53 discharges the fluid aspirated from the medical device 10 to a discharge portion (not illustrated). The liquid delivering pump 54 receives the fluid supplied from a fluid source 60, and delivers the fluid to the medical device 10. The fluid source 60 stores a saline solution (i.e., saline) as the fluid.

The control apparatus 11 further has a priming switch 56 for performing a priming start operation, and a display unit 57 for displaying a state of aspiration or liquid delivering. The display unit 57 can display the state of aspiration and liquid delivering, for example, with light using an LED.

As illustrated in FIG. 2, the control apparatus 11 has a fluid control unit 50 and a drive control unit 52. The fluid control unit 50 and the drive control unit 52 will be collectively referred to as a control unit. The drive control unit 52 is connected to each of the aspiration pump 53 and the liquid delivering pump 54 of the control apparatus 11, and a rotary motor 49 disposed in the operation unit 22 of the medical device 10, and can control rotations of the drive control unit 52. The fluid control unit 50 can control the drive control unit 52. In this manner, the fluid control unit 50 can control starting and stopping the rotation of the treatment unit 21 in the medical device 10, and can control a flow rate of aspiration performed by the aspiration pump 53 and a flow rate of liquid delivering performed by the liquid delivering pump 54 in the control apparatus 11.

The control apparatus 11 includes a power supply unit 55 that supplies power to the fluid control unit 50 and the drive control unit 52, and an input/output unit 51 that inputs and outputs signals to and from the fluid control unit 50. The priming switch 56, the display unit 57, and a bubble detector 58 that detects bubbles in the fluid from the fluid source 60 are connected to the input/output unit 51. The bubble detector 58 detects the bubbles mixed in the liquid from the fluid source 60 and a state where the liquid stored in the fluid source 60 is absent, and stops fluid control, thereby preventing air from being mixed into a blood vessel. In addition, the rotary switch 41 disposed in the operation unit 22 of the medical device 10 is connected to the fluid control unit 50.

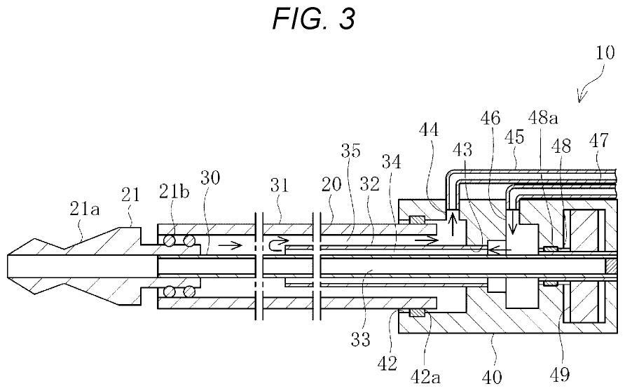

Next, a structure of the medical device 10 will be described. As illustrated in FIG. 3, the shaft portion 20 of the medical device 10 has a rotatable drive shaft 30 having the treatment unit 21 in a distal portion of the rotatable drive shaft 30, an outer sheath 31 that covers an outer side of the drive shaft 30, and an inner sheath 32 disposed inside the outer sheath 31 on the outer side of the drive shaft 30. The drive shaft 30, the inner sheath 32, and the outer sheath 31 are concentrically disposed. The drive shaft 30 internally has a guide wire lumen 33 into which a guide wire (not illustrated) is inserted. A liquid delivering lumen 34 for circulating the fluid delivered from the liquid delivering pump 54 is disposed inside the inner sheath 32, outside the drive shaft 30. An aspiration lumen 35 for circulating the fluid aspirated by the aspiration pump 53 is disposed inside the outer sheath 31, outside the inner sheath 32.

A distal portion of the treatment unit 21 has a cutting portion 21a that cuts a stenosed site or an obstructive site. A proximal portion of the treatment unit 21 extends into the outer sheath 31, is fixed to the drive shaft 30, and has a bearing portion 21b disposed on an outer peripheral side between the proximal portion and the outer sheath 31.

The drive shaft 30 has a proximal portion extending into the housing 40, and is fixed to the rotary motor 49 disposed inside the housing 40. A drive shaft holder 48 is disposed inside the housing 40 on the distal side from the rotary motor 49, and the housing 40 rotatably holds the drive shaft 30 via a seal portion 48a.

A proximal portion of the outer sheath 31 extends into the housing 40. The housing 40 has an outer sheath holder 42 that holds the outer sheath 31, and the housing 40 holds the outer sheath 31 or maintains a lumen of the outer sheath 31 in a liquid-tight manner via a seal portion 42a.

A distal portion of the inner sheath 32 is located in an intermediate portion of the outer sheath 31. Therefore, a distal end of the liquid delivering lumen 34 is located in the intermediate portion of the outer sheath 31. The fluid delivered to the liquid delivering lumen 34 flows back in a distal portion of the inner sheath 32, and flows toward the aspiration lumen 35. A proximal portion of the inner sheath 32 extends into the housing 40. The housing 40 has an inner sheath holder 43 that holds the inner sheath 32, and the proximal portion of the inner sheath 32 is fixed to the inner sheath holder 43.

The housing 40 has an aspiration port 44 to which the aspiration tube 45 is connected and a liquid delivering port 46 to which the liquid delivering tube 47 is connected. The aspiration port 44 communicates with the aspiration lumen 35 of the shaft portion 20. In addition, the liquid delivering port 46 communicates with the liquid delivering lumen 34 of the shaft portion 20.

Next, treatment performed by a medical device system according to the present embodiment will be described. In the treatment, the stenosed site inside the blood vessel is cut by the medical device 10. Prior to the use of the medical device 10, an introducer sheath (not illustrated) is percutaneously inserted into the blood vessel on an upstream side of the stenosed site of the blood vessel, and a guide wire (not illustrated) is inserted into the blood vessel via the introducer sheath.

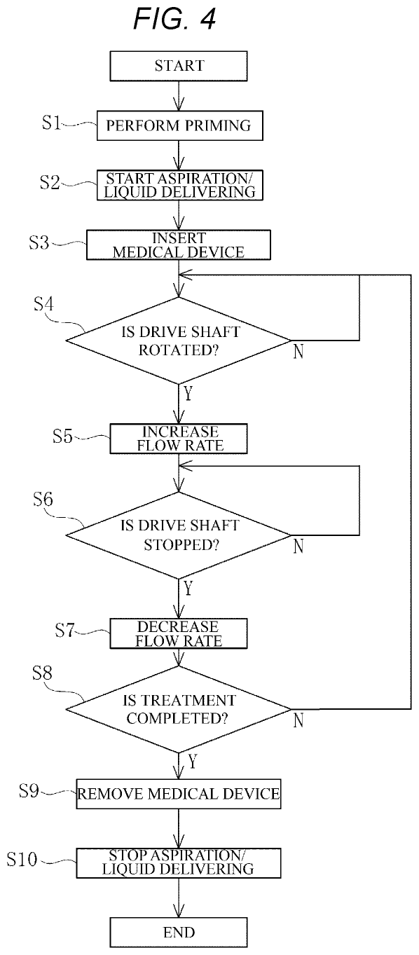

As illustrated in FIG. 4, priming is first performed on the medical device 10 (S1). The priming starts by operating the priming switch 56 of the control apparatus 11. The priming is performed through aspiration and liquid delivering for the medical device 10 from the control apparatus 11, and the medical device 10 is in a state where the shaft portion 20 is internally filled with the fluid. Even after the priming is completed, the aspiration and the liquid delivering for the shaft portion 20 are continuously performed at a low flow rate (S2). The medical device 10 is inserted into the blood vessel while the aspiration and the liquid delivering for the shaft portion 20 are maintained, and the treatment unit 21 is delivered to a target site while being guided by the guide wire.

When the treatment unit 21 is delivered to the target site, an operator operates the rotary switch 41 of the operation unit 22 to rotate the drive shaft 30 (S4). Simultaneously with starting the rotation of the drive shaft 30, the flow rate of the aspiration and the liquid delivering for the medical device 10 is increased. The treatment unit 21 is rotated by the drive shaft 30 to cut the stenosed site. A cut plaque or thrombus is drawn into the outer sheath 31 which is internally aspirated, and is discharged from the aspiration port 44 through the aspiration lumen 35. A timing at which the rotation of the drive shaft 30 starts and a timing at which the aspiration and the liquid delivering are performed on the medical device 10 do not need to be simultaneous, and either one timing may precede by a certain time.

After the stenosed site is completely cut, an operator operates the rotary switch 41 of the operation unit 22 to stop the drive shaft 30 (S6). When the drive shaft 30 is stopped, the flow rate of the aspiration and the liquid delivering for the shaft portion 20 is decreased, thereby bringing the shaft portion 20 into a state at the low flow rate (S7). Timings from when the drive shaft 30 is stopped until the aspiration and the liquid delivering are performed on the shaft portion 20 at the low flow rate may be simultaneous, or the timing may be shifted by a certain time so that a cut lesion area is discharged out from a body without any loss. In a case of treating another stenosed site, the treatment unit 21 is moved inside the blood vessel, and steps from S4 are repeated again. When the treatment is completed (S8), the medical device 10 is removed from the blood vessel (S9). Even in this case, the aspiration and the liquid delivering are continuously performed on the shaft portion 20 at the low flow rate. When the medical device 10 is removed from the living body, the aspiration and the liquid delivering for the shaft portion 20 are stopped (S10).

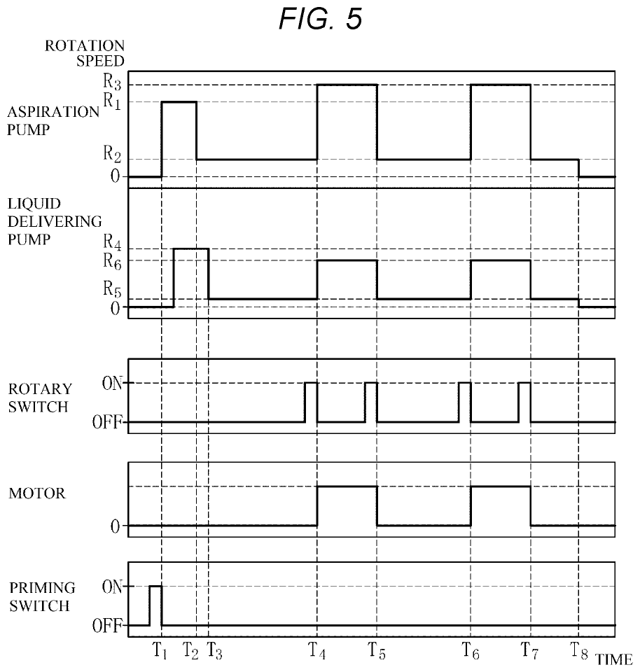

Controlling the aspiration pump 53 and the liquid delivering pump 54 by the fluid control unit 50 in the treatment will be described. As illustrated in FIG. 5, it is assumed that the priming switch 56 of the control apparatus 11 is operated at time T₁. When the priming switch 56 is operated, the fluid control unit 50 activates the aspiration pump 53, to be rotated at a rotation speed R₁. The fluid control unit 50 activates the liquid delivering pump 54 after a short delay from the activation of the aspiration pump 53 to be rotated at a rotation speed R₄. In this manner, a saline solution (i.e., saline) is injected into the medical device 10 from the liquid delivering port 46, flows inside the shaft portion 20 at a constant flow rate, and is discharged through the aspiration port 44.

The priming is automatically completed after a prescribed time elapses from when the priming switch 56 is operated. The fluid control unit 50 decreases the rotation speed of the aspiration pump 53 from R₁ to R₂ at time T₂. In addition, the fluid control unit 50 decreases the rotation speed of the liquid delivering pump 54 from R₄ to R₅ at time T₃. In this manner, a flow rate of the fluid flowing inside the shaft portion 20 is lower than that during priming. However, the fluid continuously flows at a low flow rate. In this state, the medical device 10 is inserted in S3 described above. Even while the treatment unit 21 is delivered to a target site, the fluid flows inside the shaft portion 20. Accordingly, it is possible to prevent a possibility that a thrombus may appear due to blood stagnated inside the shaft portion 20.

The rotation speed R2 of the aspiration pump 53 at the low flow rate is greater than the rotation speed R5 of the liquid delivering pump 54. Therefore, an aspiration amount of the aspiration pump 53 is greater than a liquid delivering amount of the liquid delivering pump 54, and the blood inside the blood vessel is aspirated. Since there is a blood pressure inside the blood vessel, the aspiration is performed prior to the liquid delivering. In this manner, while sealing performance may be ensured by suppressing the pressure applied to the seal portion 48a, the fluid inside the shaft portion 20 can flow. In addition, since the aspiration is performed prior to the liquid delivering, the thrombus inside the shaft portion 20 can be pushed out into the blood vessel, and a relative risk of a peripheral embolism can be reduced. In addition, here, it is premised that the aspiration pump 53 and the liquid delivering pump 54 have the same liquid transport amount per one rotation. Here, a magnitude of the aspiration amount and the liquid delivering amount corresponds to a flowing direction of the liquid inside the shaft portion 20. When the aspiration amount is greater than the liquid delivering amount, it means that the liquid flows in a proximal direction near the distal end of the shaft portion 20. On the other hand, when the liquid delivering amount is greater than the aspiration amount, it means that the liquid flows in a distal end direction near the distal end of the shaft portion 20.

When the treatment unit 21 is delivered to the target site and the operator operates the rotary switch 41 of the operation unit 22 at time T₄, as described above, the drive shaft 30 and the treatment unit 21 are rotated by the rotary motor 49. At this time, the fluid control unit 50 increases the rotation speed of the aspiration pump 53 from R₂ to R₃. In addition, the fluid control unit 50 increases the rotation speed of the liquid delivering pump 54 from R₅ to R₆. In this manner, both the flow rate of the aspiration and the flow rate of the liquid delivering inside the shaft portion 20 increase, and a plaque or a thrombus cut by the treatment unit 21 may be aspirated and discharged into the outer sheath 31.

When the cutting by the treatment unit 21 is completed and the operator operates the rotary switch 41 of the operation unit 22 at time T₅, the rotary motor 49 is stopped, and the drive shaft 30 and the treatment unit 21 are also stopped. At this time, the fluid control unit 50 decreases the rotation speed of the aspiration pump 53 from R₃ to R₂. In addition, the fluid control unit 50 decreases the rotation speed of the liquid delivering pump 54 from R₆ to R₅. In this manner, the flow rate of the fluid flowing inside the shaft portion 20 decreases. However, the fluid continuously flows at the low flow rate. In this state, the medical device 10 is removed in S9 described above, or the treatment unit 21 is disposed again inside the blood vessel. Even after the treatment is performed by the treatment unit 21, the fluid flows inside the shaft portion 20 until the medical device 10 is removed from the blood vessel. Accordingly, it is possible to prevent a possibility that the thrombus may appear due to the blood stagnated inside the shaft portion 20.

In FIG. 5, the operator operates the rotary switch 41 of the operation unit 22 again at time T₆. The drive shaft 30 and the treatment unit 21 are rotated by the rotary motor 49, and the rotation is stopped by operating the rotary switch 41 at time T₇. While the rotary motor 49 is rotated, the fluid control unit 50 increases the rotation speed of the aspiration pump 53 and the rotation speed of the liquid delivering pump 54. Thereafter, when the medical device 10 is removed from the blood vessel, at time T8, the aspiration pump 53 and the liquid delivering pump 54 are stopped, and the fluid flow in the shaft portion 20 is stopped. The liquid flow at time T₈ may be automatically stopped by the fluid control unit 50 when a sensor detects that the medical device 10 is removed from the blood vessel, or may be manually stopped by an operator or a user who performs an input operation on a switch disposed in the control apparatus 11. Although FIG. 4 illustrates a flow of the treatment for stopping the liquid flow at time T8, the fluid flow may be continued without stopping the fluid flow after the medical device 10 is removed (S9). In this case, after the medical device 10 is removed from the blood vessel, it is preferable that the distal end of the medical device 10 is continuously immersed in a container filled with saline solution (i.e., saline) or that the priming operation is performed again in this state. In this manner, it is possible to prevent a possibility that the thrombus may appear since discharging the blood remaining inside the medical device 10 to outside the medical device 10. The fluid is caused to continuously flow even after the medical device 10 is removed from the blood vessel. Accordingly, the medical device 10 is no longer complicated by a sensor, and the operator or the user does not need to make an effort to operate the medical device 10. Therefore, the system can be relatively simplified as a whole.

As described above, while the treatment unit 21 performs the treatment, the fluid control unit 50 aspirates the fluid at a high flow rate (first flow rate), and delivers the fluid at a high flow rate (third flow rate). While the treatment unit 21 does not perform the treatment, the fluid control unit 50 aspirates the fluid at a second flow rate which is lower than the first flow rate, and delivers the fluid at a fourth flow rate which is lower than the third flow rate. The second flow rate and the fourth flow rate are respectively minimum flow rates in the aspiration pump 53 and the liquid delivering pump 54. The fluid is caused to flow inside the shaft portion 20 at the low flow rate even while the treatment is not performed. Accordingly, a possibility that a thrombus may appear due to blood stagnated inside the shaft portion 20 can be prevented. The applicant of the present application has confirmed that the thrombus can be prevented from appearing inside the shaft portion 20 by setting the flow rate of the fluid in the aspiration lumen 35, for example, to 5 ml/min. However, without being limited to a flow rate of the fluid in the aspiration lumen 35, for example, to 5 ml/min, the flow rate can be, for example, 1 ml/min to 50 ml/min, and 1 ml/min to 10 ml/min, for example, in a case where an atherectomy procedure time is considered.

In the above-described treatment method, in a state where the aspiration and the liquid delivering are performed on the shaft portion 20 at the low flow rate, the shaft portion 20 starts to be inserted into the blood vessel. However, after the shaft portion 20 starts to be inserted into the blood vessel, the aspiration and the liquid delivering may start for the shaft portion 20 at the low flow rate. Specifically, the aspiration and the liquid delivering may automatically start by causing a sensor to detect a state where the shaft portion 20 is inserted into the blood vessel, or may automatically start after a first treatment operation is completed (S8). A configuration in which the sensor detects the insertion state into the blood vessel will be described later. In addition, an operation unit such as a switch may be disposed in either or both of the control apparatus 11 and the housing 40 so that the aspiration and the liquid delivering manually start by the operation of the operator or the user. In addition, after the medical device 10 is completely removed from the blood vessel, the aspiration and the liquid delivering for the shaft portion 20 at the low flow rate are stopped. However, before the medical device 10 is completely removed from the blood vessel, the aspiration and the liquid delivering for the shaft portion 20 at the low flow rate may be stopped. As in the above-described insertion, the aspiration and the liquid delivering before and after the medical device 10 is removed from the blood vessel may be automatically stopped by causing the sensor to detect the insertion state into the blood vessel, or may dispose the operation unit such as the switch in either or both of the control apparatus 11 and the housing 40 so that the aspiration and the liquid delivering are manually stopped by the operation of the operator or the user. In addition, even after the medical device 10 is completely removed from the blood vessel, the aspiration and the liquid delivering may be continuously performed on the shaft portion 20 at the low flow rate without being stopped. In any case, after the medical device 10 is removed from the blood vessel, the priming operation is performed again. In this manner, it is desirable that the blood inside the shaft portion 20 is discharged to replace the inside of the shaft portion 20 with saline solution. In this manner, even in a case where the medical device 10 is inserted into the blood vessel again, the same treatment can be repeatedly performed without weakening the aspiration force.

In addition, after the medical device 10 is removed from the blood vessel, the priming may be performed to insert the medical device 10 into the blood vessel again. Even in this case, the fluid flows at the low flow rate during a period other than the treatment period of the treatment unit 21 while the medical device 10 is inserted into the living body. Accordingly, a possibility that the thrombus may appear due to the blood stagnated inside the shaft portion 20 can be prevented. In addition, the priming may be performed in a state where the medical device 10 is present inside the body.

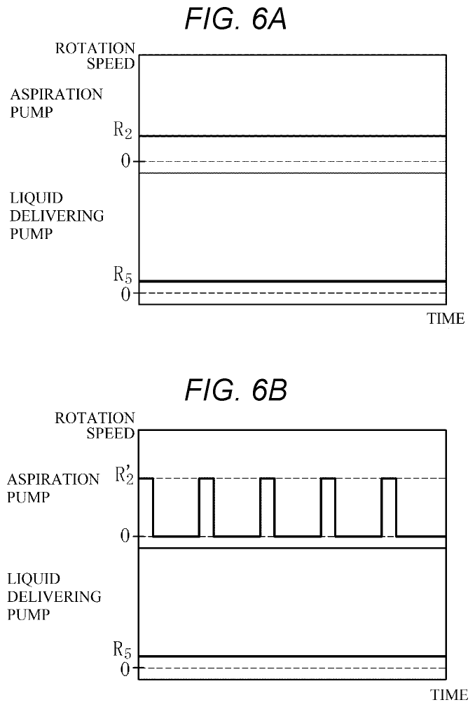

In the above-described embodiment, as illustrated in FIG. 6A, the aspiration and the liquid delivering for the shaft portion 20 at the low flow rate are operated at a constant rotation speed by both the aspiration pump 53 and the liquid delivering pump 54. On the other hand, as illustrated in FIG. 6B, while the liquid delivering pump 54 may be operated at the constant rotation speed, the aspiration pump 53 may be intermittently operated at a rotation speed R₂′. In this manner, an amount of blood loss of a patient can be reduced, and the blood or the thrombus stagnated inside the medical device 10 can be rather efficiently discharged outward of the body.

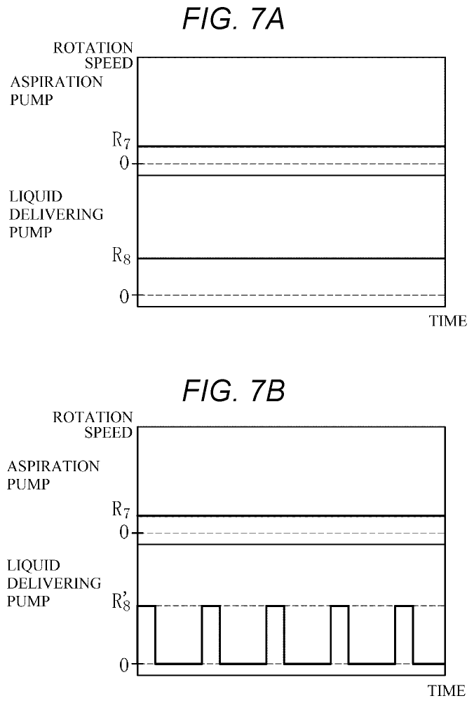

As illustrated in FIG. 7A, the rotation speed of the aspiration pump 53 may be set to R₇ lower than R₂, and the rotation speed of the liquid delivering pump 54 may be set to R₈ higher than R₅. In this manner, the liquid delivering pump 54 may have the higher rotation speed than the aspiration pump 53. In this case, the liquid delivering amount of the liquid delivering pump 54 is greater than the aspiration amount of the aspiration pump 53. Accordingly, the saline solution is injected into the blood vessel. Therefore, the amount of blood loss in the treatment can be reduced by reducing the amount of blood aspiration. In addition, the liquid delivering is performed prior to the aspiration. Accordingly, a concentration of the saline solution inside the shaft portion 20 can be increased, and the concentration of the blood can be decreased. Therefore, it is possible to further prevent the thrombus from appearing inside the shaft portion 20.

In addition, as illustrated in FIG. 7B, in a case where the liquid delivering is performed prior to the aspiration, while the aspiration pump 53 may be operated at a constant rotation speed, the liquid delivering pump 54 may be intermittently rotated at a rotation speed R₈′. In this manner, the amount of the saline solution flowing into the patient's body can be reduced.

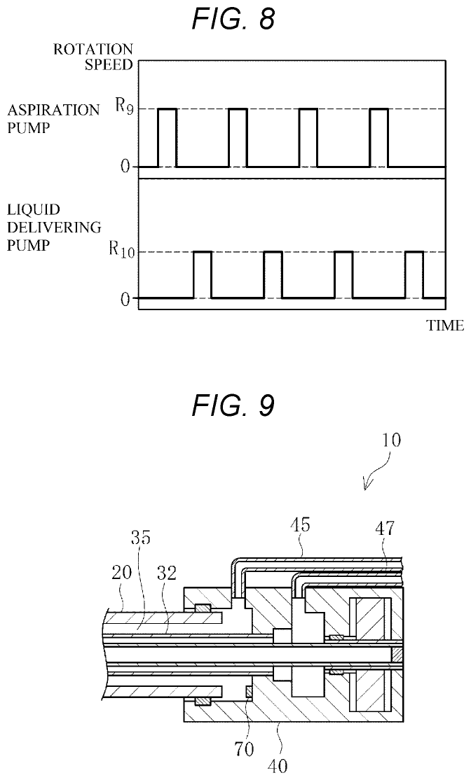

As illustrated in FIG. 8, the aspiration pump 53 may perform an intermittent operation at a rotation speed R₉, and the liquid delivering pump 54 may also perform an intermittent operation at a rotation speed R₁₀. In FIG. 8, the aspiration pump 53 and the liquid delivering pump 54 are alternately operated. However, both of these may be synchronized with each other, or may be operated at a prescribed interval. In this manner, while the amount of blood loss amount of the patient may be reduced, the amount of the saline solution flowing into the patient's body can be reduced.

The medical device 10 according to the present embodiment performs the aspiration and the liquid delivering from the blood vessel. Therefore, it is necessary to control the blood loss amount of the patient during the treatment. The control apparatus 11 may have a notification unit and an operation unit for that purpose. For example, it is possible to provide a notification unit that measures a time after the medical device 10 is inserted into the living body or a treatment time, and that uses sound or light to notify the operator when a prescribed time elapses. In addition, it is possible to provide a notification unit that measures the blood loss amount after the medical device 10 is inserted into the living body in or near the aspiration pump 53, and that uses sound or light to notify the operator when the blood loss amount exceeds a prescribed value. In addition, it is possible to provide a notification unit that measures the aspiration amount in or near the aspiration pump 53, and that changes intensity of sound or light in accordance with the aspiration amount. The above-described notification may be performed through a crystal display of a treatment time, an aspiration time other than the treatment time, and the amount of blood loss. Alternatively, the aspiration or the liquid delivering may be forcibly stopped simultaneously with the notification. In addition, when the aspiration and the liquid delivering are performed on the shaft portion 20 at the low flow rate, the control apparatus 11 or the housing 40 may emit a sound (for example, a beep sound at a prescribed interval) or light (for example, flashing light). In this manner, the operator or the user can continuously recognize a fact that the aspiration and the liquid delivering are performed on the shaft portion 20 at the low flow rate. According to the above-described method, the amount of blood loss of the patient during the procedure can be reduced.

In addition, the control apparatus 11 or the housing 40 may have a temporary stop operation unit for temporarily stopping the aspiration performed by the aspiration pump 53. In this manner, in a case where the amount of blood loss needs to be reduced during the treatment, the operator or the user can rather easily and immediately take a countermeasure by operating the temporary stop operation unit. When the aspiration is temporarily stopped by a temporary stop operation for a prescribed time, the notification may be performed using the sound or the light, or the aspiration or the liquid delivering may be forcibly restarted. In this manner, even in a case where the operator or the user forgets to restart the temporary stop operation, the thrombus can be avoided from appearing.

In the above-described treatment method, in order to perform the aspiration and the liquid delivering on the shaft portion 20 at the low flow rate by causing a sensor unit 70 to detect an insertion state of the shaft portion 20 into the blood vessel, as illustrated in FIG. 9, the sensor unit 70 including a pressure sensor can be disposed inside the housing 40 of the operation unit 22. The sensor unit 70 is disposed at a position for communicating with the aspiration lumen 35. When the shaft portion 20 is inserted into an artery, a blood pressure caused by a reversed blood flow (i.e., a flow of blood from the blood vessel into the housing 40 of the operation unit) is applied into the shaft portion 20. The blood pressure is detected by the sensor unit 70. Accordingly, it is possible to detect that the shaft portion 20 is inserted into the blood vessel. When the sensor unit 70 detects that the shaft portion 20 is inserted into the blood vessel, as described above, the fluid control unit 50 starts the aspiration and the liquid delivering for the shaft portion 20 at the low flow rate. In addition, when the sensor unit 70 detects a pressure decrease in the blood pressure, the fluid control unit 50 determines that the shaft portion 20 has been removed from the blood vessel, stops the aspiration and the liquid delivering for the shaft portion 20 at the low flow rate.

A position of the sensor unit 70 serving as the pressure sensor is not limited to an example in FIG. 9, and may be any position where the blood pressure caused by the reversed blood flow can be detected. In addition, in FIG. 9, the blood pressure from the blood vessel is detected by the sensor unit 70 via the aspiration lumen 35. However, a pressure detecting lumen may be separately disposed in the shaft portion 20 or another shaft member, and the pressure may be detected by the sensor unit 70 via the lumen.

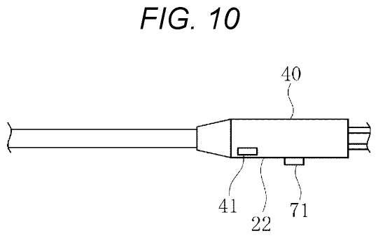

As illustrated in FIG. 10, the sensor unit 71 may be formed of a push button switch extending outward on an outer portion of the operation unit 22. The sensor unit 71 can detect whether or not the operation unit 22 is placed on a base or a holder. In a case where the sensor unit 71 detects that the operation unit 22 is placed on the base, it may be determined that the medical device 10 is not inserted into the blood vessel, and the fluid control unit 50 may not perform the aspiration and the liquid delivering on the shaft portion 20. In a case where the sensor unit 71 detects that the operation unit 22 is separated from the base, it may be determined that the medical device 10 is inserted into the blood vessel, and the fluid control unit 50 may start to perform the aspiration and the liquid delivering on the shaft portion 20.

As described above, according to the present embodiment, the control apparatus 11 of the elongated tubular medical device 10 is configured to be inserted into the living body having the distal portion in which the treatment unit 21 for treating the object in the biological lumen is disposed includes the aspiration source 53 or the liquid delivering source 54 connected to the medical device 10, and the control unit 50 that controls the flow rate of the fluid flowing inside the medical device 10. The control unit 50 performs any one of the following operations. In a case where the aspiration source 53 is connected to the medical device 10, the control unit 50 controls the aspiration source 53 so that the fluid inside the medical device 10 is aspirated at the first flow rate during the treatment period of the treatment unit 21, and so that the fluid inside the medical device 10 is aspirated at the second flow rate lower than the first flow rate during the period other than the treatment period, while the medical device 10 is inserted into the living body. Alternatively, in a case where the liquid delivering source 54 is connected to the medical device 10, the control unit 50 controls the liquid delivering source 54 so that the fluid is delivered into the medical device 10 at the third flow rate during the treatment period of the treatment unit 21, and so that the fluid is delivered into the medical device 10 at the fourth flow rate lower than the third flow rate during the period other than the treatment period, while the medical device 10 is inserted into the living body. In this manner, the fluid flows inside the medical device 10 during the period other than the treatment period, while the medical device 10 is inserted into the living body. Accordingly, the thrombus can be prevented from appearing inside the medical device 10.

In addition, according to the present embodiment, the control apparatus 11 of the elongated tubular medical device 10 is configured to be inserted into the living body and having the distal portion in which the treatment unit 21 for treating the object in the biological lumen is disposed includes the aspiration source 53 connected to the medical device 10, the liquid delivering source 54 connected to the medical device 10, and the control unit 50 that controls the flow rate of the fluid flowing inside the medical device 10. The control unit 50 performs the following operations. In a case where the aspiration source 53 is connected to the medical device 10, the control unit 50 controls the aspiration source 53 so that the fluid inside the medical device 10 is aspirated at the first flow rate during the treatment period of the treatment unit 21, and so that the fluid inside the medical device 10 is aspirated at the second flow rate lower than the first flow rate during the period other than the treatment period, while the medical device 10 is inserted into the living body. Alternatively, in a case where the liquid delivering source 54 is connected to the medical device 10, the control unit 50 controls the liquid delivering source 54 so that the fluid is delivered into the medical device 10 at the third flow rate during the treatment period of the treatment unit 21, and so that the fluid is delivered into the medical device 10 at the fourth flow rate lower than the third flow rate during the period other than the treatment period, while the medical device 10 is inserted into the living body. In this manner, the fluid flows inside the medical device 10 during the period other than the treatment period, while the medical device 10 is inserted into the living body. Accordingly, the thrombus can be prevented from appearing inside the medical device 10.

In addition, the control unit 50 can control the aspiration source 53 and the liquid delivering source 54 so that the second flow rate is greater than the fourth flow rate. In this manner, the aspiration is performed prior to the liquid delivering. Accordingly, while sealing performance is ensured for the drive shaft 30, the fluid can be caused to flow inside the medical device 10. In addition, since the aspiration is performed prior to the liquid delivering, the thrombus inside the medical device 10 cannot be pushed out into the blood vessel.

In addition, the control unit 50 can control the aspiration source 53 and the liquid delivering source 54 so that the fourth flow rate is greater than the second flow rate. In this manner, since the liquid delivering is performed prior to the aspiration, the blood loss amount in the treatment can be suppressed by reducing the blood aspiration amount. In addition, the liquid delivering is performed prior to the aspiration. Accordingly, concentration of the saline solution inside the medical device 10 can be increased, and concentration of the blood can be decreased. Therefore, it is possible to further prevent the thrombus from appearing inside the medical device 10.

In addition, in a case where the aspiration source 53 is connected to the medical device 10, the control unit 50 can cause the aspiration source 53 to aspirate the fluid at a flow rate greater than the second flow rate when the priming is performed on the medical device 10. In this manner, while the priming time of the medical device 10 is shortened, the control unit 50 can cause the fluid to flow inside the medical device 10 at the low flow rate during the period other than the treatment period.

In addition, in a case where the liquid delivering source 54 is connected to the medical device 10, the control unit 50 can cause the liquid delivering source 54 to deliver the fluid at the fourth flow rate when the priming is performed on the medical device 10. In this manner, while the priming time of the medical device 10 is shortened, the control unit 50 can cause the fluid to flow inside the medical device 10 at the low flow rate during the period other than the treatment period.

In addition, during the period other than the treatment period, while the medical device 10 is inserted into the living body, the control unit 50 can rotate the medical device 10 at a lower speed than during the treatment period. In this manner, the fluid inside the medical device 10 can be caused to flow by the low speed rotation of the medical device 10. The fluid flow inside the medical device 10 can be promoted during the period other than the treatment period, and the thrombus can be prevented from appearing.

In addition, the medical device 10 further has the sensor unit 70 that detects the insertion state of the medical device 10 into the living body. In a case where the aspiration source 53 is connected to the medical device 10, the control unit 50 can cause the fluid inside the shaft portion 20 to flow at the second flow rate during the period other than the treatment period, while the sensor unit 70 detects the state where the medical device 10 is inserted into the living body. In this case, while the medical device 10 is inserted into the living body, the control unit 50 can cause the fluid to automatically flow at the second flow rate.

In addition, the medical device 10 further has the sensor unit 70 that detects the insertion state of the medical device 10 into the living body. In a case where the liquid delivering source 54 is connected to the medical device 10, the control unit 50 can cause the fluid inside the medical device 10 to flow at the fourth flow rate during the period other than the treatment period, while the sensor unit 70 detects the state where the medical device 10 is inserted into the living body. In this case, while the medical device 10 is inserted into the living body, the control unit 50 can cause the fluid to automatically flow at the fourth flow rate.

In addition, the medical device 10 further has the temporary stop operation unit that temporarily stops the aspiration in a case where the aspiration source 53 is connected to the medical device 10, when the control unit 50 causes the aspiration source 53 to aspirate the fluid inside the medical device 10 at the second flow rate. In this manner, in a case where the blood loss amount needs to be reduced during the treatment, the operator or the user can rather easily and immediately take a countermeasure by operating the temporary stop operation unit.

In addition, the medical device 10 further has the temporary stop operation unit that temporarily stops the liquid delivering in a case where the liquid delivering source 54 is connected to the medical device 10, when the control unit 50 causes the liquid delivering source 54 to deliver the fluid into the medical device 10 at the fourth flow rate. In this manner, in a case where the blood loss amount needs to be reduced during the treatment, the operator or the user can rather easily and immediately take a countermeasure by operating the temporary stop operation unit.

In addition, the medical device 10 further has the notification unit that uses the sound or the light to notify the operator as follows. In a case where the aspiration source 53 is connected to the medical device 10, the operator is notified that the control unit 50 is causing the fluid inside the medical device 10 to be aspirated at the second flow rate, or in a case where the liquid delivering source 54 is connected to the medical device 10, the operator is notified that the control unit 50 is delivering the fluid into the medical device 10 at the fourth flow rate. In this case, the notification unit can be used in controlling the amount of blood loss of the patient.

In addition, according to the present embodiment, the method of controlling the medical device system including the elongated tubular shaft portion 20 that can be inserted into the living body, the medical device 10 having the treatment unit 21 disposed in the distal portion of the shaft portion 20, and the fluid control unit 50 that controls the flow rate of the fluid flowing inside the shaft portion 20. In the method of controlling the medical device system, the fluid control unit 50 causes the fluid to flow inside the shaft portion 20 at the constant flow rate until the treatment unit 21 starts the treatment after the shaft portion 20 is inserted into the living body. The fluid control unit 50 causes the fluid to flow inside the shaft portion 20 at the flow rate greater than the constant flow rate until the treatment unit 21 completes the treatment after starting the treatment. The fluid control unit 50 causes the fluid to flow inside the shaft portion 20 at the constant flow rate until the shaft portion 20 is removed from the living body after the treatment unit 21 completes the treatment. In this manner, the fluid flows inside the shaft portion 20 during the period other than the treatment period, while the shaft portion 20 is inserted into the living body. Accordingly, the thrombus can be prevented from appearing inside the shaft portion 20. The fluid flow during the period other than the treatment period may be realized through automatic control, for example, performed by software, or may be realized by causing the sensor to detect a state where the medical device 10 is present inside the body. Alternatively, the fluid flow during the period other than the treatment period may be realized by disposing the operation unit in either or both of the control apparatus 11 or the housing 40, and by the operator or the user operating the operation unit.

In addition, the method of controlling the flow rate of the fluid according to the present embodiment includes connecting the liquid delivering source 54 for delivering the fluid into the medical device 10 having the treatment unit 21 disposed in the distal portion to the medical device 10, or connecting the aspiration source 53 for aspirating the fluid to the medical device 10, actuating the control unit 50 that controls the flow rate of the fluid to cause the fluid to flow in the liquid delivering source 54 or the aspiration source 53 at a constant flow rate while the medical device 10 is stopped, and actuating the control unit to cause the fluid to flow in the liquid delivering source 54 or the aspiration source 53 at a flow rate greater than the constant flow rate while the medical device 10 is actuated. In this manner, even while the medical device 10 is stopped, the liquid delivering source 54 or the aspiration source 53 causes the fluid to flow at the constant flow rate. Accordingly, the thrombus can be prevented from appearing inside the medical device 10.

The present disclosure is not limited to the above-described embodiments, and various modifications can be made by those skilled in the art within the technical idea of the present disclosure. For example, in the above-described embodiment, the aspiration pump 53 performs the aspiration, and the liquid delivering pump 54 performs the liquid delivering. However, the other means may perform the aspiration or the liquid delivering. For example, the aspiration or the liquid delivering may be performed using a syringe. In addition, with regard to the aspiration, the blood may be discharged outward through the inside of the shaft portion 20 by using the blood pressure inside the blood vessel.

In addition, in the above-described embodiment, the fluid control unit 50 controls the rotation speed of the aspiration pump 53 and the liquid delivering pump 54 to control a difference between the aspiration amount and the liquid delivering amount while the treatment is not performed by the treatment unit 21. However, the difference between the aspiration amount and the liquid delivering amount may be controlled by other means. For example, the aspiration pump 53 and the liquid delivering pump 54 may have the same rotation speed. The difference between the aspiration amount and the liquid delivering amount while the treatment is not performed by the treatment unit 21 may be controlled using a difference between the inner diameters of the aspiration tube 45 and the liquid delivering tube 47. In addition, the difference between the aspiration amount and the liquid delivering amount may be controlled by the aspiration pump 53 and the liquid delivering pump 54, which have different fluid discharge amounts per unit time. In addition, a combination of the difference between the inner diameters of the aspiration tube 45 and the liquid delivering tube 47 and aspiration amount and the liquid delivering amount may be adopted.

In addition, the drive shaft 30 may be rotated at a relatively low speed while the treatment is not performed by the treatment unit 21. The drive shaft 30 can be formed as a coiled tubular body having a prescribed winding direction. In this case, an axial flow in one direction can be generated around the drive shaft 30 by rotating the drive shaft 30 in one direction. During the period other than the treatment period of the treatment unit 21 while the shaft portion 20 is inserted into the living body, the drive shaft 30 is rotated in one direction at the lower speed than during the treatment period. A flow in an aspiration direction can be generated inside the shaft portion 20 so that the movement of the fluid can be promoted by the axial flow. In addition, the drive shaft 30 is rotated in an opposite direction of the above-described one direction. In this manner, a flow in a liquid delivering direction can be generated inside the shaft portion 20 so that the movement of the fluid can be further promoted by the liquid delivering. Furthermore, the rotation of the drive shaft 30 in one direction and the rotation in the opposite direction may be alternately performed at a prescribed cycle. In this manner, the flow in the aspiration direction and the flow in the liquid delivering direction can be alternately generated inside the shaft portion 20.

In addition, in the above-described embodiment, the fluid control unit 50 performs both the aspiration and the liquid delivering on the shaft portion 20. However, any one of the aspiration and the liquid delivering may be performed. In addition, in the above-described embodiment, the shaft portion 20 has the drive shaft 30, and has the aspiration lumen 35 and the liquid delivering lumen 34. However, the shaft portion 20 may have only one lumen used for the aspiration or the liquid delivering. In addition, the shaft portion 20 may have the aspiration lumen 35 and the liquid delivering lumen 34, and may not have the drive shaft 30. In addition, in the above-described embodiment, the target for preventing the thrombus is not limited to the shaft portion 20. The thrombus can be prevented from appearing by causing the fluid to flow in the housing 40 and the aspiration tube 45. The present disclosure is also applicable to medical devices other than the atherectomy device as in the above-described embodiment.

The detailed description above describes embodiments of a control apparatus for a medical device that causes a fluid to flow in an elongated tubular medical device configured to be inserted into a living body, and a method of controlling a flow rate of a fluid. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A control apparatus for an elongated tubular medical device configured to be inserted into a living body, the elongated tubular medical device having a distal portion in which a treatment unit configured to treat an object in a biological lumen is disposed, the control apparatus comprising:
an aspiration source or a liquid delivering source connected to the medical device;
a control unit configured to control a flow rate of a fluid flowing inside the medical device; and
wherein in a case where the aspiration source is connected to the medical device, the control unit is configured to control the aspiration source so that the fluid inside the medical device is aspirated at a first flow rate during a treatment period of the treatment unit, and so that the fluid inside the medical device is aspirated at a second flow rate lower than the first flow rate during a period other than the treatment period, while the medical device is inserted into the living body, or
wherein in a case where the liquid delivering source is connected to the medical device, the control unit is configured to control the liquid delivering source so that the fluid is delivered into the medical device at a third flow rate during the treatment period of the treatment unit, and so that the fluid is delivered into the medical device at a fourth flow rate lower than the third flow rate during the period other than the treatment period, while the medical device is inserted into the living body.

2. The control apparatus according to claim 1, wherein in the case where the aspiration source is connected to the medical device, when priming is performed on the medical device, the control unit is configured to cause the aspiration source to aspirate the fluid at a flow rate higher than the second flow rate.

3. The control apparatus according to claim 1, wherein in the case where the liquid delivering source is connected to the medical device, when priming is performed on the medical device, the control unit is configured to cause the liquid delivering source to deliver the fluid at a flow rate higher than the fourth flow rate.

4. The control apparatus according to claim 1, wherein during the period other than the treatment period, while the medical device is inserted into the living body, the control unit is configured to rotate the medical device at a lower speed than during the treatment period.

5. The control apparatus according to claim 1, further comprising:
a sensor unit configured to detect a state where the medical device is inserted into the living body; and
wherein in the case where the aspiration source is connected to the medical device, the control unit is configured to cause the fluid inside the medical device to flow at the second flow rate during the period other than the treatment period, while the sensor unit detects the state where the medical device is inserted into the living body.

6. The control apparatus according to claim 1, further comprising:
a sensor unit configured to detect a state where the medical device is inserted into the living body; and
wherein in the case where the liquid delivering source is connected to the medical device, the control unit is configured to cause the fluid inside the medical device to flow at the fourth flow rate during the period other than the treatment period, while the sensor unit detects the state where the medical device is inserted into the living body.

7. The control apparatus according to claim 1, further comprising:
a temporary stop operation unit configured to temporarily stop the aspiration, in the case where the aspiration source is connected to the medical device, when the control unit causes the aspiration source to aspirate the fluid inside the medical device at the second flow rate.

8. The control apparatus according to claim 1, further comprising:
a temporary stop operation unit configured to temporarily stop the liquid delivering, in the case where the liquid delivering source is connected to the medical device, when the control unit causes the liquid delivering source to deliver the fluid into the medical device at the fourth flow rate.

9. The control apparatus according to claim 1, further comprising:
a notification unit configured to issue a notification by sound or light that the control unit is causing the aspiration source to aspirate the fluid inside the medical device at the second flow rate in the case where the aspiration source is connected to the medical device, or to issue a notification that the control unit is causing the liquid delivering source to deliver the fluid into the medical device at the fourth flow rate in the case where the liquid delivering source is connected to the medical device.

10. A control apparatus for an elongated tubular medical device configured to be inserted into a living body, the elongated tubular medical device having a distal portion in which a treatment unit configured to treat an object in a biological lumen is disposed, the control apparatus comprising:
an aspiration source connected to the medical device;
a liquid delivering source connected to the medical device;
a control unit configured to control a flow rate of a fluid flowing inside the medical device; and
wherein the control unit is configured to control the aspiration source so that the fluid inside the medical device is aspirated at a first flow rate during a treatment period of the treatment unit, and so that the fluid inside the medical device is aspirated at a second flow rate lower than the first flow rate during a period other than the treatment period, while the medical device is inserted into the living body, and
wherein the control unit is configured to control the liquid delivering source so that the fluid is delivered into the medical device at a third flow rate during the treatment period of the treatment unit, and so that the fluid is delivered into the medical device at a fourth flow rate lower than the third flow rate during the period other than the treatment period, while the medical device is inserted into the living body.

11. The control apparatus according to claim 10, wherein the control unit is configured to control the aspiration source and the liquid delivering source so that the second flow rate is higher than the fourth flow rate.

12. The control apparatus according to claim 10, wherein the control unit is configured to control the aspiration source and the liquid delivering source so that the fourth flow rate is higher than the second flow rate.

13. The control apparatus according to claim 10, wherein in a case where the aspiration source is connected to the medical device, when priming is performed on the medical device, the control unit is configured to cause the aspiration source to aspirate the fluid at a flow rate higher than the second flow rate.

14. The control apparatus according to claim 10, wherein in a case where the liquid delivering source is connected to the medical device, when priming is performed on the medical device, the control unit is configured to cause the liquid delivering source to deliver the fluid at a flow rate higher than the fourth flow rate.

15. The control apparatus according to claim 10, wherein during the period other than the treatment period, while the medical device is inserted into the living body, the control unit is configured to rotate the medical device at a lower speed than during the treatment period.

16. The control apparatus according to claim 10, further comprising:
a sensor unit that detects a state where the medical device is inserted into the living body; and
wherein in a case where the aspiration source is connected to the medical device, the control unit is configured to cause the fluid inside the medical device to flow at the second flow rate during the period other than the treatment period, while the sensor unit detects the state where the medical device is inserted into the living body.

17. The control apparatus according to claim 10, further comprising:
a sensor unit that detects a state where the medical device is inserted into the living body; and
wherein in a case where the liquid delivering source is connected to the medical device, the control unit is configured to cause the fluid inside the medical device to flow at the fourth flow rate during the period other than the treatment period, while the sensor unit detects the state where the medical device is inserted into the living body.

18. The control apparatus according to claim 10, further comprising:
a temporary stop operation unit configured to temporarily stop the aspiration, in a case where the aspiration source is connected to the medical device, when the control unit causes the aspiration source to aspirate the fluid inside the medical device at the second flow rate; or
a temporary stop operation unit configured to temporarily stop the liquid delivering, in a case where the liquid delivering source is connected to the medical device, when the control unit causes the liquid delivering source to deliver the fluid into the medical device at the fourth flow rate.

19. The control apparatus according to claim 10, further comprising:
a notification unit configured to issue a notification by sound or light that the control unit is causing the aspiration source to aspirate the fluid inside the medical device at the second flow rate in a case where the aspiration source is connected to the medical device, or to issue a notification that the control unit is causing the liquid delivering source to deliver the fluid into the medical device at the fourth flow rate in a case where the liquid delivering source is connected to the medical device.

20. A method of controlling a flow rate of a fluid, the method comprising:
connecting a liquid delivering source to a medical device, the liquid delivering source configured to deliver the fluid into the medical device having a treatment unit disposed in a distal portion, or connecting an aspiration source configured to aspirate the fluid to the medical device;
actuating a control unit configured to control a flow rate of the fluid to cause the fluid to flow in the liquid delivering source or the aspiration source at a constant flow rate while the medical device is stopped; and
actuating the control unit to cause the fluid to flow in the liquid delivering source or the aspiration source at a flow rate higher than the constant flow rate while the medical device is actuated.
